(19) United States
(12) Patent Application Publication
(10) Pub. No.: US 2025/0000593 A1
(43) Pub. Date: Jan. 2, 2025

(54) ROBOTIC ARM SYSTEM AND SURGICAL SYSTEM

(71) Applicant: BEIJING HURWA MEDICAL TECHNOLOGY CO., LTD., Beijing (CN)

(72) Inventors: Jinbang GUO, Beijing (CN); Zhao ZHANG, Beijing (CN)

(73) Assignee: BEIJING HURWA MEDICAL TECHNOLOGY CO., LTD., Beijing (CN)

(21) Appl. No.: 18/883,187

(22) Filed: Sep. 12, 2024

Related U.S. Application Data
(63) Continuation of application No. PCT/CN2023/103931, filed on Jun. 29, 2023.

(30) Foreign Application Priority Data
Jul. 1, 2022 (CN) 202210770053.6

(51) Int. Cl. A61B 34/30; A61B 34/20; A61B 90/50; B25J 9/00

(52) U.S. Cl. CPC A61B 34/30; A61B 34/20; A61B 90/50; B25J 9/0009

(57) ABSTRACT

Disclosed are a robotic arm system and a surgical system. The robotic arm system includes a robotic arm and a motor driver. The robotic arm is provided with a base end and an end, the end is used to connect an electric tool for surgery, and the base end is used to fix the robotic arm. The motor driver is disposed on the end of the robotic arm, and the motor driver is configured to convert constant current from a power source into a variable current required by the electric tool. The robotic arm system is configured to form an electric connection between the electric tool and the motor driver when the electric tool is connected to the end.

### CROSS-REFERENCE TO RELATED APPLICATIONS

The present application is a continuation of International Application No. PCT/CN2023/103931, filed on Jun. 29, 2023, which claims priority to Chinese Patent Application No. 202210770053.6, filed on Jul. 1, 2022. The disclosures of the aforementioned applications are hereby incorporated by reference in their entireties.

### TECHNICAL FIELD

The present disclosure relates to the field of medical equipment, and in particular, to a robotic arm system and a surgical system.

### BACKGROUND

Among orthopedic surgery systems assisted by a robot, a mechanical positioning structure carried by a robotic arm is provided by some systems, and a surgeon holds an electric tool such as an electric swing saw or an electric file to operate a bone under guidance of the mechanical positioning structure. A cooperative robotic arm is used in other systems, the robotic arm carries an electric tool and provides guidance for the electric tool, and a surgeon may directly push the electric tool or an end of the robotic arm to operate a bone.

Generally, a robotic arm system in a surgical system includes a robotic arm and a trolley. The robotic arm has a complex electro-mechanical structure, which requires supporting accessories such as a control module and the like. The trolley is used to carry the robotic arm and the supporting accessories. An electric tool does not require a lot of power, so that a direct current (DC) motor is mostly applied to the electric tool. Among DC motors, a brushless DC motor has better performance than a brushed DC motor, so it is widely used. However, the brushless DC motor cannot directly use a constant current provided by a DC power source, and it needs to use a pulsating DC current formed after being processed by a driving module. In addition, normal operation of the brushless DC motor also requires the control module to receive a control signal to form the required pulsating DC current. In a general application environment, a driving module of a machine using the brushless DC motor may be more centrally assembled with the motor, in this way, the use of a cable between the motor and the driving module may be reduced, which is beneficial to reducing inductance loss and electromagnetic interference caused by transmission of the pulsating DC current.

However, there are other limitations in the application of the brushless DC motor in the field of surgery. For example, a surgical tool requires to be sterilized before surgery, but the sterilized (high temperature sterilization or plasma sterilization and the like) environments may not be an ideal work environment for components of a driving module or control module. Therefore, integrating the driving module and the control module with the surgical tool will cause their electrical components to undergo a harsh sterilized environment. The above problems also exist in a surgical system using a robot. It is known that a current solution is to separate the driving module and the control module from the motor, and both of them are disposed away from the electric tool. In this way, a volume of the electric tool is not increased, and the driving module does not need to be sterilized. Of course, this configuration makes the above-mentioned advantages of an integrated assembly manner (such as low inductance loss) no longer exist.

### SUMMARY

The present disclosure provides a robotic arm system and a surgical system, to at least solve one of the technical problems in the related art to some extent.

In a first aspect, the present disclosure provides a robotic arm system, including a robotic arm and a motor driver. The robotic arm is provided with a base end and an end, the end is used to connect an electric tool for surgery, and the base end is used to fix the robotic arm. The motor driver is disposed on the end of the robotic arm, and the motor driver is configured to convert a constant current from a power source into a variable current required by the electric tool. The robotic arm system is configured to form an electric connection between the electric tool and the motor driver when the electric tool is connected to the end.

In a first possible implementation, a current pathway of the constant current is located inside the robotic arm.

In combination with the foregoing possible implementations, in a second possible implementation, the electric connection between the electric tool and the motor driver is located on a joint interface of the electric tool and the end of the robotic arm.

In combination with the foregoing possible implementations, in a third possible implementation, the robotic arm system further includes a transition apparatus for connecting the electric tool to the end of the robotic arm, the transition apparatus is disposed at the end of the robotic arm, and the motor driver is disposed in the transition apparatus.

In combination with the foregoing possible implementations, in a fourth possible implementation, the transition apparatus is provided with a metal housing.

In combination with the foregoing possible implementations, in a fifth possible implementation, the transition apparatus is configured so that: a current pathway is formed between the motor driver and the power source when the transition apparatus is connected with the robotic arm; and a current pathway is formed between the motor driver and the electric tool when the transition apparatus is connected with the electric tool.

In combination with the foregoing possible implementations, in a sixth possible implementation, the transition apparatus is detachably connected with the robotic arm.

In combination with the foregoing possible implementations, in a seventh possible implementation, the transition apparatus is configured to be detachably connected with the electric tool.

In combination with the foregoing possible implementations, in an eighth possible implementation, the transition apparatus includes a flange assembly, the flange assembly includes a first flange and a second flange, the first flange is connected with the second flange, the first flange is used to connect with the robotic arm, and the second flange is used to connect with the electric tool.

In combination with the foregoing possible implementations, in a ninth possible implementation, the motor driver is disposed between the first flange and the second flange.

In combination with the foregoing possible implementations, in a tenth possible implementation, the motor driver includes a first electrical interface and a second electrical interface, the first electrical interface is used to connect with the power source, and the second electrical interface is used to connect with the electric tool.

In combination with the foregoing possible implementations, in an eleventh possible implementation, an electric connection between the first electrical interface and the power source and/or an electric connection between the second electrical interface and the electric tool are spring contact structures or contact pin structures.

In combination with the foregoing possible implementations, in a twelfth possible implementation, the motor driver includes an adapter module, an input module, a driving module, a control module and an output module, the adapter module is used to connect with the power source, and the output module is used to connect with the electric tool.

In combination with the foregoing possible implementations, in a thirteenth possible implementation, the adapter module, the input module, the driving module, the control module and the output module are connected in a plug-in manner.

In combination with the foregoing possible implementations, in a fourteenth possible implementation, an electrical interface is disposed at the end of the robotic arm, an end of the electrical interface is used to connect with the power source, and the other end of the electrical interface is connected with the motor driver.

In a second aspect, the present disclosure provides a surgical system, including the robotic arm as described in the first aspect, a navigation system and a control system. The navigation system is configured to measure an orientation of the electric tool. The control system is configured to acquire the orientation of the electric tool from the navigation system and drive the robotic arm to move the electric tool to a target position according to a surgical plan.

According to the robotic arm system proposed by the present disclosure, as the configuration of the motor driver, a length of a current pathway between the motor driver and the motor is reduced, thereby inductance loss and external electromagnetic interference is reduced. Moreover, a requirement of sterilization for the surgical system in clinical use is also met, that is, since the motor driver is disposed on the robotic arm, and the electric tool is separated from the motor driver when the power tool is detached from the robotic arm, so it is not necessary to sterilize the motor driver.

### BRIEF DESCRIPTION OF THE DRAWINGS

FIG. 1 is a structural schematic diagram of a surgical system according to an embodiment of the present disclosure.

FIG. 2 is a structural schematic diagram of a robotic arm system according to an embodiment of the present disclosure.

FIG. 3 is a structural schematic diagram of an electric tool according to an embodiment of the present disclosure.

FIG. 4 is a structural schematic diagram I of a transition apparatus according to an embodiment of the present disclosure.

FIG. 5 is a section view of a transition apparatus according to an embodiment of the present disclosure.

FIG. 6 is a structural schematic diagram of another transition apparatus according to an embodiment of the present disclosure.

FIG. 7 is a structural schematic diagram of a motor driver according to an embodiment of the present disclosure.

FIG. 8 is a structural schematic diagram of an adapter module according to an embodiment of the present disclosure.

FIG. 9 is a schematic diagram I of a motor driver disposed in a transition apparatus according to an embodiment of the present disclosure.

FIG. 10 is a schematic diagram II of the motor driver disposed in the transition apparatus according to an embodiment of the present disclosure.

FIG. 11 is a schematic diagram of a joint structure between an electric tool and a robotic arm according to an embodiment of the present disclosure.

FIG. 12 is a schematic diagram of a joint structure between an electric tool and a robotic arm according to an embodiment of the present disclosure.

FIG. 13 is a schematic diagram of a joint structure between an electric tool and a robotic arm according to an embodiment of the present disclosure.

FIG. 14 is a schematic diagram of a flange structure according to an embodiment of the present disclosure.

FIG. 15 is a schematic diagram of another flange structure according to an embodiment of the present disclosure.

### DETAILED DESCRIPTION OF THE EMBODIMENTS

Hereinafter, features and exemplary embodiments of various aspects of the present disclosure will be described in detail. In order to make purposes, technical solutions and advantages of the present disclosure clearer and more understandable, the present disclosure will be further described in detail in combination with the drawings and specific embodiments as below. It should be understood that, the specific embodiments as described herein are intended merely to explain the present disclosure, rather than limiting the present disclosure. For a person skilled in the art, the present disclosure may be implemented without some of the specific details. The description for the embodiments as below is intended merely to provide better understanding to the present disclosure by illustrating examples of the present disclosure.

It should be noted that, in the present specification, the relation terms, such as “first” and “second” or the like, are used only to distinguish one entity or operation from another entity or operation, rather than requiring or implying that there is any actual relationship or order between these entities or operations. Moreover, the terms “comprise”, “include” or any other variations thereof is intended to cover non-exclusive inclusion, such that any process, method, item or device including a series of elements not only includes said elements, but also further includes other elements not specifically listed, or further includes the elements inherent to the process, method, item or device. In the cases without any more limitations, the element as defined by a sentence of “include . . . ” will not exclude the case in which the process, method, item or device including said elements further includes additional same element.

It should be noted that, the embodiments and the features therein in the present disclosure may be combined with one another, unless it causes contradiction. Hereinafter, the embodiments will be described in detail in combination with the drawings.

The present disclosure provides a robotic arm system for connecting an electric tool for surgery. The robotic arm system includes a robotic arm, a power source, a motor driver and a trolley. The robotic arm has a base end and an end, the base end is connected to the trolley, and the end is used for connecting an electric tool. The power source is disposed on a side (such as in the trolley) of the robotic arm where the base end is located, and is configured to output a constant current. The motor driver is disposed on the end of the robotic arm for driving a motor of the electric tool. The motor driver is configured to convert the constant current into a variable current required by the electric tool. The motor driver is disposed on the end of the robotic arm, when the electric tool is connected to the end of the robotic arm, the electric tool also electrically communicates with the motor driver. In this way, a length of a current pathway between the motor driver and the motor is very short, so that electromagnetic radiation and interference, and inductance loss is weakened. In addition, when the electric tool is separated from the end of the robotic arm, it may also be separated from the motor driver and cut off the electrical connection at the same time, so that the electric tool is capable to be sterilized separately, and thus the motor driver does not need to enter in a harsh sterilized environment with the electric tool.

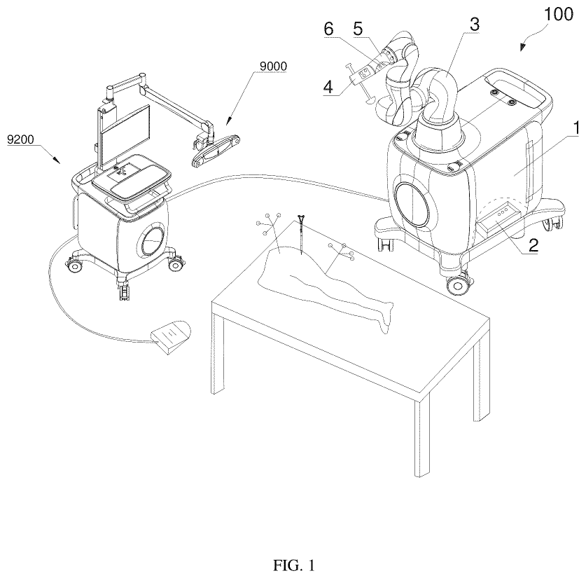

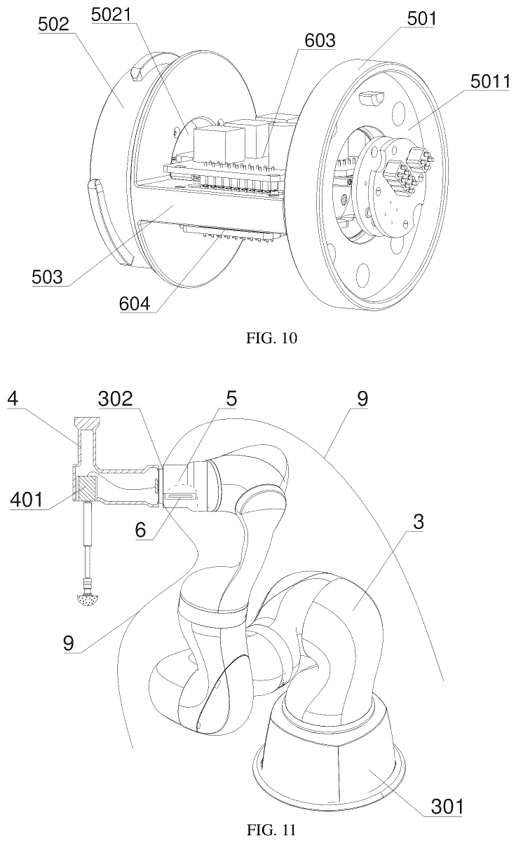

Specifically, as shown in FIGS. 1 to 11, the robotic arm system includes a trolley body 1, a power source 2, a robotic arm 3, an electric tool 4, a transition apparatus 5 and a motor driver 6.

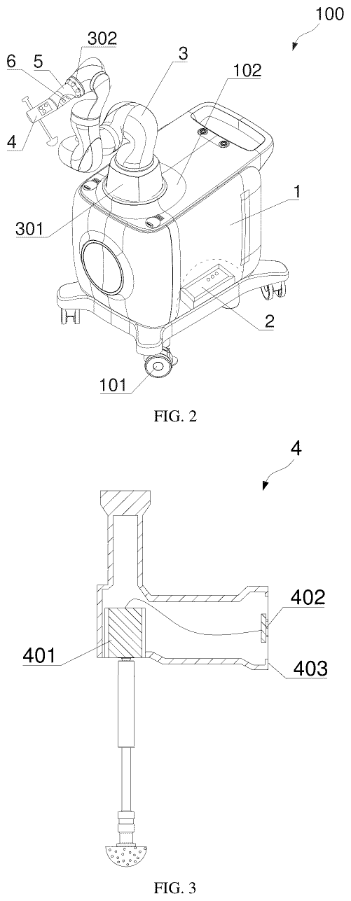

Specifically, referring to FIG. 1 and FIG. 2, the trolley body 1 is a packaged frame structure, which is roughly a cube. The trolley body 1 includes wheels 101 and a robotic arm connector 102. The wheels 101 are disposed at a lower portion of the trolley body 1 for moving and transferring the robotic arm system 100. The robotic arm connector 102 is disposed at an upper portion of the trolley for connecting to the robotic arm 3. In addition, the trolley body 1 has a relatively large volume and weight, and as a base of the robotic arm 3, the gravity center of the whole robotic arm system 100 is stably located on the trolley body 1. When the robotic arm 3 acts, the trolley body 1 provides stable support for the robotic arm 3.

The power source 2 is disposed in the trolley body 1. The power source 2 is configured to output a constant direct current. In the present embodiment, the current output by the power source 2 is a direct current with 24V.

The robotic arm 3 has a plurality of movable joints, and is capable of assisting/replacing a human hand to move the electric tool to a target position within a surgical space. Both ends of the robotic arm 3 are a base end 301 and an end 302, respectively, and the base end 301 is a fixed end. When the robotic arm 3 acts, the base end 301 maintains stationary, and the end 302 may move within the surgical space. The base end 301 of the robotic arm 3 is fixed to the robotic arm connector 102 of the trolley body 1.

As shown in FIG. 3, the electric tool 4 is an actuator carrying a surgical tool, which mainly includes a housing, a motor 401 and a surgical tool. The electric tool 4 is provided with an electrical interface 402 and a flange connector 403. The motor 401 is a direct current (DC) brushless motor. The electrical interface 402 and the flange connector 403 are disposed at a same position which is at a same end face of the housing, as shown in FIG. 3. A current pathway between the electrical interface 402 and the motor 401 is inside the electric tool 4, for example the electrical interface 402 is connected to the motor 401 by a cable. The flange connector 403 is used to connect the electric tool 4 to the end 302 of the robotic arm 3. As shown in FIG. 1, in hip surgery, for preparation of an acetabulum, the surgical tool is an acetabular file. The surgical tool, driven by the motor 401, may be rotated at a high speed, to grind out a predetermined shape for installing a prosthesis. In knee surgery, the surgical tool is a swing saw, and the swing saw swings at a high speed under the drive of the motor to cut a target plane. Certainly, the electric tool 4 is not limited to the actuator for the preparation of acetabulum and the actuator for the knee osteotomy described above, but may also be other electric tools carrying a surgical tool, and this is not specifically limited herein.

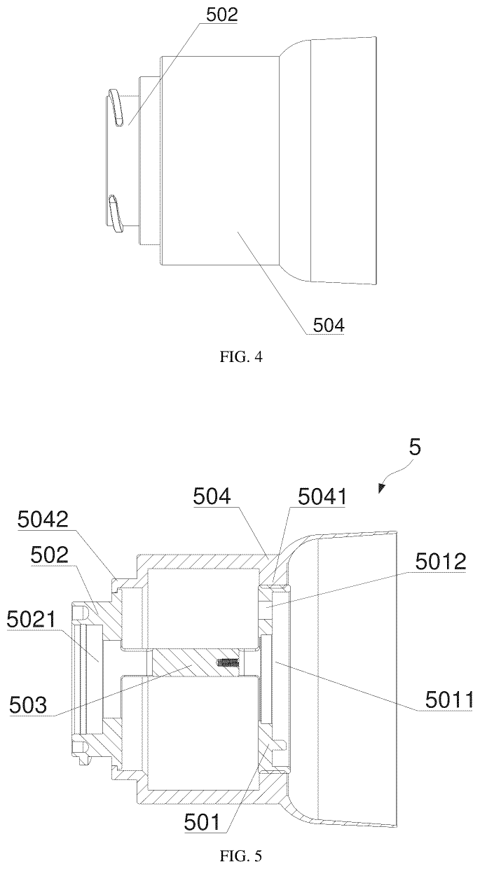

As shown in FIG. 4 and FIG. 5, the transition apparatus 5 is a flange structure with a housing, which includes a first flange 501, a second flange 502, a connecting section 503 and a metal housing 504. The first flange 501 and the second flange 502 are both annular, and there are a first accommodating space 5011 and a second accommodating space 5021 in the ring bodies thereof, respectively. The first flange 501 and the second flange 502 are connected through the connecting section 503. The connecting section 503 is plate-shaped, and is vertically connected with the first flange 501 and the second flange 502 to form a flange structure with an “H”-shaped cross section. The first flange 501 is provided with a through hole 5012, and the through hole 5012 is used for a bolt to pass through to connect with the robotic arm 3. An outer periphery of the first flange 501 is provided with thread. The metal housing 504 is sleeve-shaped, and is sleeved on outer peripheries of the first flange 501, the second flange 502 and the connecting section 503.

Specifically, in the present embodiment, two ends of the metal housing 504 are opened, and the metal housing 504 includes a first connecting part 5041 and a second connecting part 5042. The first connecting part 5041 is provided with thread, and the second connecting part 5042 is provided with a retainer flange. When assembling, the metal housing 504 is sleeved on an outer periphery of the flange structure along a direction from the second flange 502 to the first flange 501. By rotating the metal housing 504, the first connecting part 5041 is in threaded connection with the first flange, and continues rotating the metal housing 504 until the retainer flange contacts with the second flange 502 to form a limit at this position, so that the metal housing 504 and the flange structure are fixed. As shown in FIG. 6, in an optional implementation, an anti-loosening pin 7 is disposed on an end of the second connecting part 5042. The anti-loosening pin 7 is configured to press the flange structure and the retainer flange, and a pressing direction is parallel to an axial direction of the metal housing 504. In this way, the flange structure may be further fixed with the metal housing 504 by the anti-loosening pin 7, to prevent the flange structure from detaching from the metal housing 504 when the threaded connection between the first connecting part 5041 and the first flange 501 is loose. Certainly, the anti-loosening pin 7 may also be replaced by a screw, a rivet or a retainer ring or the like.

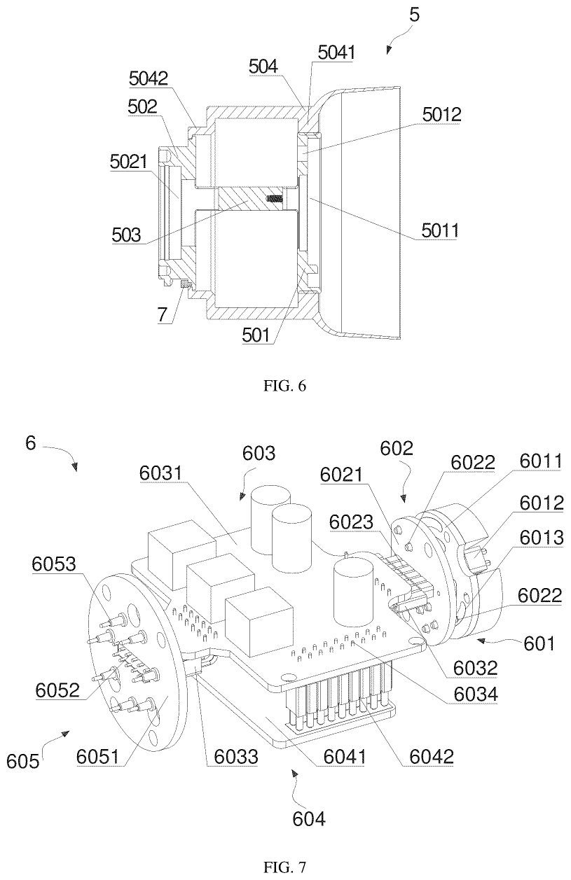

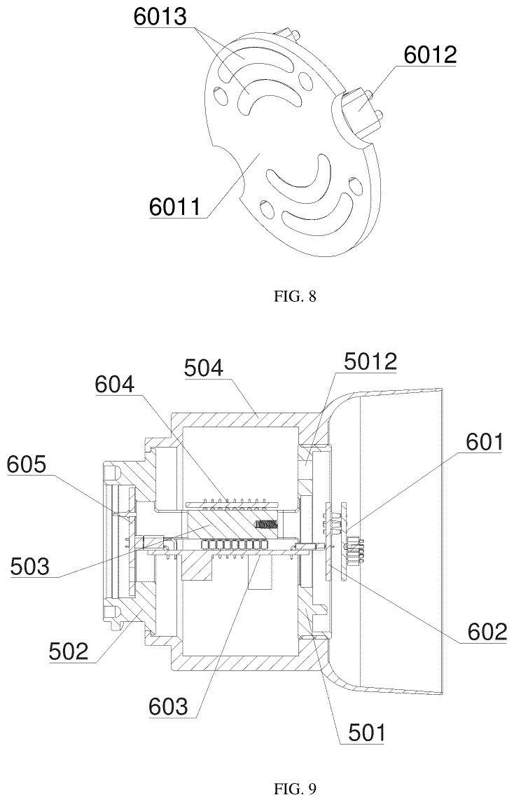

The configuration of the motor driver 6 is shown in FIG. 7 and FIG. 8. The motor driver 6 is used to drive the motor 401 of the electric tool 4. In the present embodiment, the motor driver 6 is disposed in the transition apparatus 5. The motor driver 6 includes an adapter module 601, an input module 602, a driving module 603, a control module 604 and an output module 605. The adapter module 601, the input module 602, the driving module 603, the control module 604 and the output module 605 are connected in sequence, and each module is a Printed Circuit Board (PCB) board with contact pins and/or contacts.

The adapter module 601 includes an adapter circuit board 6011, an adapter spring pin 6012 and an adapter contact 6013. The adapter circuit board 6011 is roughly circular. The adapter circuit board 6011 is provided with a mounting hole and a positioning groove for fixing the adapter module 601. The adapter spring pin 6012 and the adapter contact 6013 communicates with each other through the adapter circuit board 6011. The adapter spring pin 6012 is a metal spring pin, which is disposed on a side of the adapter circuit board 6011, and is used to connect with the power source 2. The adapter contact 6013 is disposed on the other side of the adapter circuit board 6011, the adapter contact 6013 includes two groups of arc-shaped contacts with different radii, the number of the adapter contacts 6013 in each group is two, and the two groups of the adapter contacts 6013 are oppositely disposed at intervals of 180 degrees. The adapter contact 6013 is embedded in the adapter circuit board 6011, and the surface of the adapter contact 6013 is flush with the surface of the adapter circuit board 6011. In an optional implementation, the number of the adapter contacts 6013 is not limited to the configuration of two groups with the number of four, which may be increased or decreased according to the number of interfaces which need to adapter coupling, and this is not limited herein.

The input module 602 includes an input circuit board 6021, an input contact pin 6022 and an input spring pin 6023. The input circuit board 6021 is circular, the input contact pin 6022 and the input spring pin 6023 are disposed on two sides of the input circuit board 6021, respectively, and the input contact pin 6022 and the input spring pin 6023 communicates with each other through the input circuit board 6021. There are two groups of input spring pins 6023 concentrically distributed on the input circuit board 6021, which are used to connect with the adapter contacts 6013 correspondingly. The input contact pins 6022 are arranged in a single row.

The driving module 603 includes a driving circuit board 6031, a first driving slot 6032, a second driving slot 6033 and a driving contact pin 6034. The first driving slot 6032 and the second driving slot 6033 are located at both ends of the driving circuit board 6031. The driving contact pin 6034 is located at a side of the driving circuit board 6031. The first driving slot 6032 is used to connect with the input contact pin 6022 of the input module 602. The driving circuit board 6031 includes a Pulse-Width Modulation (PWM) module, and the PWM module is used to convert a direct current with 24V into a variable pulse current, to control operation of a direct current motor with 24V.

The control module 604 includes a control circuit board 6041 and a control slot 6042. The control circuit board 6041 is used to assist the driving module 603 in converting a constant direct current into a PWM current. The control slot 6042 is used to connect with the driving contact pin 6034. After the control circuit board 6041 communicates with the driving circuit board 6031, the control circuit board 6041 transmits a control signal to the driving circuit board 6031, to make the driving circuit board 6031 generate a 24V variable pulse direct current with a corresponding frequency to drive the direct current motor. The variable pulse direct current is used to control a stator of a direct current brushless motor to generate a variable magnetic field, and then drives a rotor of a permanent magnet to rotate.

The output module 605 includes an output circuit board 6051, output contact pins 6052 and output spring pins 6053. The output contact pins 6052 and the output spring pins 6053 are located at two sides of the output circuit board 6051, respectively, and the output contact pins 6052 and the output spring pins 6053 communicates with each other through the output circuit board 6051. The output contact pins 6052 are used to connect with the second driving slot 6033, and the output spring pins 6053 are used to connect with the motor 401 to transmit a current to the motor 401.

The input module 602, the driving module 603, the control module 604 and the output module 605 form a main body of the motor driver 6. When assembling, the input contact pin 6022 is connected with the first driving slot 6032, the driving contact pin 6034 is connected with the control slot 6042, and the second driving slot 6033 is connected with the output contact pin 6052. That is, two sides of the driving module 603 are connected with the input module 602 and the output module 605, respectively. The control module 604 is connected to the driving module 603, and the control module 604 and the driving module 603 are capable to communicate with each other. Specifically, control information of the 24V direct current and the motor is transmitted through the adapter module and the input module in sequence. The direct current acts on the driving module 603, the control information is transmitted to the control module 604 through the driving module 603, and a corresponding control signal is transmitted to the driving module 603 by the control module 604 according to the control information, to make the driving module 603 convert the direct current with 24V into the variable pulse direct current with 24V. The variable pulse direct current with 24V is output through the output module 605.

In the present embodiment, as shown in FIGS. 9 to 11, the motor driver 6 is disposed in the transition apparatus 5. The input module 602 is fixed in the first accommodating space 5011 through a screw. The output module 605 is fixed in the second accommodating space 5021 through a screw. The driving module 603 and the control module 604 are fixed to two sides of the connecting section 503 through screws, respectively. In addition, when each module is connected with the flange structure of the transition apparatus 5, there is an insulation pad between each module and the flange structure, to avoid short circuit. The motor driver 6 is packaged in the transition apparatus 5. The metal housing 504 of the transition apparatus 5 forms a protection for the motor driver 6, which not only avoids collision damage and contamination of dust and bloody sputter to the motor driver 6, but also forms a shielding to a certain extent, and thus the electromagnetic interference caused by the outside to the motor driver is reduced 6.

Continuing to refer to FIGS. 1 to 11, in the configuration of the robotic arm system, the power source 2 is disposed inside the trolley body 1. The cable built-in the robotic arm 3 extends from the power source 2 to the end 302 of the robotic arm 3, and a power interface is formed on the end 302 of the robotic arm 3. The adapter module 601 is also disposed at the end 302, and the adapter module 601 is connected to the end 302 of the robotic arm 3 through the mounting hole and the positioning slot. The power interface is connected with the adapter spring pins 6012 of the adapter module 601. In this way, the adapter contacts 6013 of the adapter module 601 forms an interface capable of outputting a constant direct current. Moreover, the cable is built in the robotic arm, which will not cause the winding and interference of the cable to the robotic arm and electric tool the when moving the robotic arm and using and moving the electric tool in the surgical space.

The robotic arm is an electromechanical product with high-precision, which is difficult to develop and relatively high in cost. A medical instrument company generally does not develop and produce a robotic arm by itself, but purchases a ready-made robotic arm and integrates it into a surgical robot system. An electrical interface for supplying power to load is reserved on an end of this robotic arm, and a power supply circuit with a constant current is correspondingly disposed inside the robotic arm. In the technical solution of the present disclosure, since the motor driver 6 is disposed on the end 302 of the robotic arm 3, there is the constant current in the power source in the trolley and the driver, so that the power supply circuit reserved by the robotic arm may be used for supplying power, without disposing a cable outside the robotic arm.

In the present embodiment, the electric tool 4 is connected to the end 302 of the robotic arm 3 through the transition apparatus 5. Specifically, the first flange 501 of the transition apparatus 5 is connected with the end 302 of the robotic arm 3 through a bolt, and the second flange 502 of the transition apparatus 5 is connected to the electric tool 4 through a flange connector. The foregoing two connections are both detachable connections. In addition, when the first flange 501 is connected with the end 302 of the robotic arm 3, the input spring pins 6023 of the input module 602 contact with the adapter contacts 6013 of the adapter module 601 to form a pathway. When the second flange 502 is connected with the flange connector 403 of the electric tool 4, the output spring pins 6053 of the output module 605 is connected with the electrical interface 402 of the electric tool 4. In this way, with the configuration of the transition apparatus 5, the electric tool 4 is fixed to the end 302 of the robotic arm 3 in a mechanical connection manner, and the synchronous connections of the motor driver 6 with the power source 2 and the motor 401 is also realized, thereby forming a current pathway with an order of the power source 2, the motor driver 6 and the motor 401. The motor 401 may be rotated under power input of the power source 2 and under the drive of the motor driver 6.

Through the above-mentioned configuration, the motor driver 6 is disposed on the end 302 of the robotic arm 3, and a distance between the motor driver 6 and the motor 401 is relatively short. A pathway of a current control signal output by the motor driver 6 to control the rotation of the motor 401 is relatively short, so that interference of an external electromagnetic field to the current control signal is reduced as much as possible, and the inductance loss is also reduced. In addition, there is the constant direct current in the power source 2 and in the circuit of the motor driver 6, which does not generate inductance loss and electromagnetic interference to other electronic devices in the robotic arm 3. Moreover, the pathway of the current control signal is located inside the electric tool 4 and the transition apparatus 5, and the electric tool 4 and the metal housing of the transition apparatus 5 form shielding protection for the current pathway to a certain extent. In addition, in adapting to a requirement of clinical surgical, since the motor driver 6 is arranged separately from the motor 401, the motor driver 6 is not disposed inside the electric tool 4 and integrated with the motor 401, thereby a volume of the electric tool 4 is reduced. The electric tool 4 generally acts directly on a body of a patient, it is more appropriate for the electric tool 4 to drive the surgical to have a smaller volume, so that a surgeon has a relatively large field of view and operating space.

During surgery, an incision may be formed on an affected area to expose the tissue of the affected area, it is necessary to ensure a sterile environment in the surgical space, and the electric tool 4 directly acting on the patient needs to be sterilized before the surgery. But the sterilization (high temperature sterilization or plasma sterilization) environment has a higher requirement for electronic components in the motor driver 6 to meet the harsh environment. Since the motor driver 6 is arranged separately from the motor 401 and is disposed on the end 302 of the robotic arm 3, merely the surgical tool may be detached for sterilizing without sterilizing the motor driver 6. Certainly, when the motor driver 6 is not sterilized, other measures are still needed to ensure the sterile environment in an operating room. For example, the robotic arm 3 and the transition apparatus having the motor driver 6 are covered by a sterile sleeve film 9. Compared with the solution that an electric tool is directly connected with a robotic and a motor driver is detachably disposed inside an electric tool, in this way, not only the requirements of sterilization is met, but also tedious steps of disassembling the driver when the electric tool is sterilized each time and re-assembling the driver when it is used are reduced, and the volume of the electric tool 4 will not be increased.

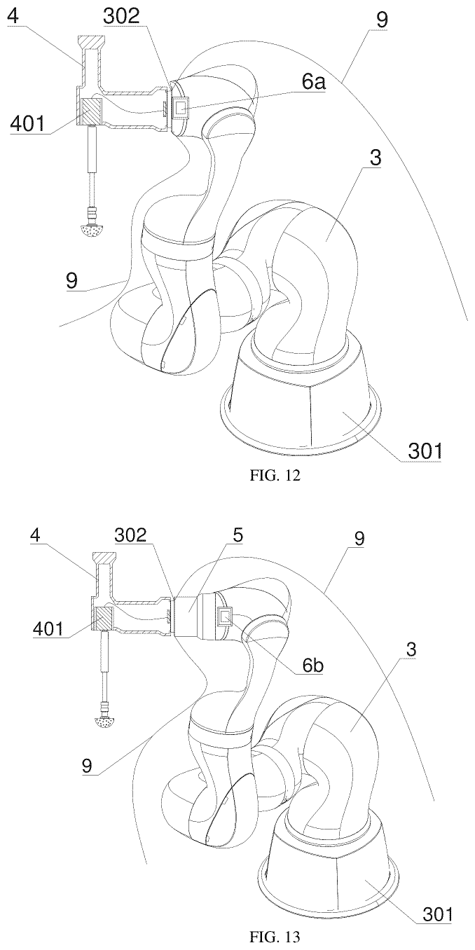

As shown in FIG. 12, in an optional implementation, the transition apparatus 5 may not be provided, the electric tool 4 is directly connected with the end 302 of the robotic arm 3 through the flange connector 403, and a motor driver 6a is disposed on the end 302 of the robotic arm 3 after being packaged by the housing likewise. The same as the foregoing embodiment, the power source 2 extends to the end 302 of the robotic arm 3 through a cable, and a power interface is formed at the end 302 of the robotic arm 3. The adapter module 601 of the motor driver 6a is connected to the power interface, and the output module 605 of the motor driver 6a is connected to the motor.

It can be understood that, through this configuration, a distance between the motor driver 6a and the motor 401 is also relatively short, and a pathway of the current control signal output by the motor driver 6a for controlling the rotation of the motor 401 is relatively short, so that the interference of the external electromagnetic field to the current control signal is reduced. The motor driver 6a is arranged separately from the motor 401 without integrating with the electric tool 4, thereby a volume of the electric tool is reduced. In addition, in order to meet the requirement of high-temperature sterilization for the electric tool, it is not necessary to disassemble and assemble the motor driver 6a, and it is only necessary to sleeve the motor driver 6 in the sterile sleeve film 9.

As shown in FIG. 13, in an optional implementation, when the transition apparatus 5 is provided, a motor driver 6b may not be disposed inside the transition apparatus 5, but be disposed separately on the end 302 of the robotic arm 3. In this way, since the motor driver 6b is disposed on the end 302 of the robotic arm 3, it is not integrated with the motor 401, but a current pathway between the motor driver 6b and the motor 401 is relatively short, so that a current control signal transmitted therebetween is not easy to be subject to or generate more electromagnetic interference, and thus the precision of motion control of the electric 401 is ensured. At the same time, the advantages in meeting the aspect of the clinical requirements have been described in detail above, and will not be repeated herein.

In an optional implementation, the configuration of the contact and the spring pin in the adapter module 601 and the input module 602 may be interchanged, that is, the adapter module 601 is provided with the spring pins, and the input module 602 is provided with the contacts. In this way, the adapter module 601 and the input module 602 are connected by the spring pins and the contacts.

In an optional implementation, the output spring pins 6053 of the output module 605 may be replaced in the form of contacts, and correspondingly, the electrical interface on the electric tool 4 is provided in the form of spring pins. In another optional implementation, the adapter spring pins 6012 on the adapter module 601 may be replaced in the form of contacts, and correspondingly, the power interface on the end 302 of the robotic arm 3 is provided in the form of spring pin.

In an optional implementation, the connection modes of the motor driver 6 with the power supply interface and the electrical interface 402 on the electric tool 4 are not limited to the forms of the contacts and the spring pins, for example, the connection mode may be also a form of plug and socket and a form of contact pin and slot, and the like.

In an optional implementation, in the connection modes among the input module 602, the control module 604, the driving module 603 and the output module 605, the configuration of the contact pins and the pin slots may be interchanged, that is, the original slot may be changed into the form of a contact pin, and the original contact pin may be changed into the form of a pin slot. In another optional embodiment, the input module 602, the control module 604, the driving module 603 and the output module 605 may be connected through an elastic sheet contact, a spring contact, a wire, a flat cable, welding, or the like.

In an optional implementation, in the motor driver 6, the input module 602, the control module 604, the driving module 603 and the output module 605 may be sequentially connected.

In an optional implementation, in the motor driver 6, the input module 602, the driving module 603, the control module 604 and the output module 605 may be sequentially connected.

In an optional implementation, in the motor driver 6, the control module 604 and the driving module 603 are connected with the output module 605, and the input module 602 is connected with the control module 604.

In an optional implementation, in the motor driver 6, the control module 604 and the driving module 603 are connected with the output module 605, and the input module 602 is connected with the driving module 603.

In an optional implementation, in the motor driver 6, the control module 604 and the driving module 603 are connected with the input module 602, and the output module 605 is connected with the control module 604.

In an optional implementation, in the motor driver 6, the control module 604 and the driving module 603 are connected with the input module 602, and the output module 605 is connected with the driving module 603.

In an optional implementation, in the motor driver 6, the connection of the input module 602, the control module 604, the driving module 603, and the output module 605 with the flange structure in the transition apparatus may be replaced by other connection modes such as rivet, strap, tie, bonding, or integral potting from a screw connection mode.

In an optional implementation, in the transition apparatus 5, a connection mode between the metal housing 504 and the flange structure is a compression-fitting joint in a mode of interference fit.

In an optional implementation, in the transition apparatus 5, the connection mode between the metal housing 504 and the flange structure is rivet joint by a rivet or joint by pressing in a pin.

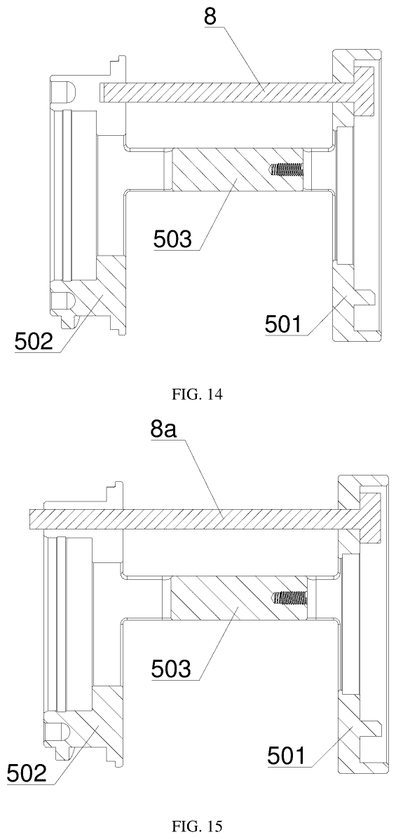

As shown in FIG. 14, in an optional implementation, the transition apparatus 5 is further provided with a “T”-shaped positioning pin 8, the “T”-shaped positioning pin 8 is connected with the first flange 501 and the second flange 502, one end of the “T”-shaped positioning pin 8 abuts against the first flange 501, and the other end of the “T”-shaped positioning pin 8 is in threaded connection with the second flange 502 and does not extend out of the second flange 502. With this configuration, strength and rigidity of the flange structure is enhanced by the “T”-shaped positioning pin 8. Moreover, the number of the “T”-shaped positioning pin 8 may be one or more. It may be understood that, the more the number of the “T”-shaped positioning pins 8 is, the higher the strength and rigidity of the flange structure is.

As shown in FIG. 15, on the basis of the foregoing configuration of the “T”-shaped positioning pin 8, in an optional implementation, an end of the “T”-shaped positioning pin 8 abuts against the first flange 501, and the other end of the “T”-shaped positioning pin 8 is in threaded connection with the second flange 502 and extends out of the second flange 502. With this configuration, the strength and rigidity of the flange structure is enhanced by the “T”-shaped positioning pin 8a, and the “T”-shaped locating pin extending out of the second flange 502 may also implement positioning and guidance for a workpiece to be connected with the second flange 502, so that the second flange 502 and the corresponding workpiece are capable to be aligned in a correct position for subsequent installation.

The present disclosure further provides a surgical system, including a robotic arm system, an electric tool 4, a navigation system 9000, and a control system 9200. The robotic arm system is the robotic arm system 100 described in the foregoing embodiment. The electric tool 4 is connected with an end of the robotic arm 3. The navigation system is configured to measure a position of the electric tool 4 The control system 9200 may obtain spatial orientation of the electric tool 4 through the navigation system 9000. The system 9200 is configured to drive the robotic arm 3 to move the electric tool 4 to a target position according to a surgical plan.

The robotic arm 3 may not only completely control the orientation of the electric tool 4 actively, but also limit a portion of degrees of freedom or a range of movement of the electric tool 4 in a cooperative mode. Specifically, the robotic arm 3 may be controlled by programming of the control system, to make the robotic arm 3 completely move autonomously according to a surgical plan, or by providing a tactile feedback or a force feedback to restrict a surgeon from manually moving the electric tool 4 beyond a predetermined virtual boundary, or provides a virtual guidance to guide the surgeon to move along some degree of freedom. The virtual boundary and the virtual guidance may come from the surgical plan, or they may be set by an input apparatus during surgery. The electric tool 4 is detachably connected with the robotic arm 3.

The navigation system 9000 is used to measure positions of the electric tool 4 and a patient. The navigation system 9000 generally includes a locator and tracers. The tracers are mounted on an actuator, a surgical tool and the patient. The tracer is generally an array formed by a plurality of tracer elements, each tracer element may send an optical or electromagnetic signal and the like in an active or passive manner. The locator (such as a binocular camera) is used to measure the orientation of the above-mentioned tracer by a three dimensional (3D) measurement technology. The locator may be a Polaris Vega® type Optical Measurement System sold by the Northern Digital Inc. (NDI) of Canada.

The control system 9200 compares a current position and a target position of the electric tool 4 according to position information acquired by the navigation system 9000, and drives the robotic arm 3 to move a prosthesis installing actuator to the target location according to a surgical plan. The surgical plan may include a moving path and moving boundary and the like of the robotic arm 3 and the like. The surgical plan is loaded on a 3D reconstruction digital model of an affected bone of a patient, which will login or be registered with tissue of the patient during the surgery.

Under the assistance of the navigation system 9000, the control system 9200 obtains the position information of the electric tool 4 relative to the patient, and controls the robotic arm 3 to move the electric tool 4 to the target position according to the surgical plan. When the surgical system performs the surgery, the interference of an external electromagnetic field to the current control signal is reduced. Moreover, the electric tool 4 and the metal housing of the transition apparatus 5 form shielding protection for a current pathway to a certain extent. In addition, in adapting to a requirement of clinical surgical, since the motor driver 6 is arranged separately from the motor 401, the motor driver 6 is not disposed inside the electric tool 4 and integrated with the motor 401, thereby a volume of the electric tool 4 is reduced. The electric tool 4 generally acts directly on a body of a patient, it is more appropriate for the electric tool 4 to drive the surgical to have a smaller volume, so that a surgeon has a relatively large field of view and operating space. Moreover, merely the surgical tool may be detached for sterilizing, without sterilizing the motor driver 6. The above obtained beneficial effects are specifically described in the first aspect of the embodiments of the present disclosure, and will not be described herein.

Though the present disclosure has been described in detail as above with the general description and specific embodiments, it is possible to make some modification or improvement thereto based on the present disclosure, which will be obvious to a person skilled in the art. Therefore, such modification or improvement made based thereon, without departing from the spirit of the present disclosure, will fall within the protection scope of the present disclosure.

What is claimed is:

1. A robotic arm system, comprising:
a robotic arm with a base end and an end, the end being used to connect an electric tool for surgery, and the base end being used to fix the robotic arm; and
a motor driver disposed on the end of the robotic arm, the motor driver being configured to convert a constant current from a power source into a variable current required by the electric tool,
wherein the robotic arm system is configured to form an electric connection between the electric tool and the motor driver when the electric tool is connected to the end.

2. The robotic arm system according to claim 1, wherein a current pathway of the constant current is located inside the robotic arm.

3. The robotic arm system according to claim 1, wherein the electric connection between the electric tool and the motor driver is located on a joint interface of the electric tool and the end of the robotic arm.

4. The robotic arm system according to claim 1, wherein the robotic arm system further comprises a transition apparatus for connecting the electric tool to the end of the robotic arm, the transition apparatus is disposed on the end of the robotic arm, and the motor driver is disposed in the transition apparatus.

5. The robotic arm system according to claim 4, wherein the transition apparatus has a metal housing.

6. The robotic arm system according to claim 4, wherein the transition apparatus is configured so that: a current pathway is formed between the motor driver and the power source when the transition apparatus is connected with the robotic arm; and a current pathway is formed between the motor driver and the electric tool when the transition apparatus is connected with the electric tool.

7. The robotic arm system according to claim 6, wherein the transition apparatus is detachably connected with the robotic arm.

8. The robotic arm system according to claim 6, wherein the transition apparatus is configured to be detachably connected with the electric tool.

9. The robotic arm system according to claim 6, wherein the transition apparatus comprises a flange assembly, the flange assembly comprises a first flange and a second flange, the first flange is connected with the second flange, the first flange is used to connect with the robotic arm, and the second flange is used to connect with the electric tool.

10. The robotic arm system according to claim 9, wherein the motor driver is disposed between the first flange and the second flange.

11. The robotic arm system according to claim 10, wherein both ends of the metal housing are opened, and the metal housing comprises a first connecting part and a second connecting part, the first connecting part is provided with thread, the second connecting part is provided with a retainer flange, the first connecting part is configured to be in threaded connection with the first flange, and the retainer flange is configured to form a limit to the second flange.

12. The robotic arm system according to claim 11, wherein an anti-loosening member is disposed on an end of the second connecting part, and the anti-loosening member is configured to press the flange assembly and the retainer flange together in a direction parallel to an axial direction of the metal housing.

13. The robotic arm system according to claim 9, wherein the transition apparatus is further provided with a “T”-shaped positioning pin, the “T”-shaped positioning pin is connected with the first flange and the second flange, one end of the T-shaped positioning pin abuts against the first flange, and the other end of the T-shaped positioning pin is in threaded connection.

14. The robotic arm system according to claim 1, wherein the motor driver comprises a first electrical interface and a second electrical interface, the first electrical interface is used to connect with the power source, and the second electrical interface is used to connect with the electric tool.

15. The robotic arm system according to claim 14, wherein an electric connection between the first electrical interface and the power source is a spring contact structure or a contact pin structure.

16. The robotic arm system according to claim 14, wherein an electric connection between the second electrical connector and the electric tool is a spring contact structure or a contact pin structure.

17. The robotic arm system according to claim 1, wherein the motor driver comprises an adapter module, an input module, a driving module, a control module and an output module, the adapter module is used to connect with the power source, and the output module is used to connect with the electric tool.

18. The robotic arm system according to claim 17, wherein the adapter module, the input module, the driving module, the control module and the output module are connected in a plug-in manner.

19. The robotic arm system according to claim 1, wherein an electrical interface is disposed at the end of the robotic arm, an end of the electrical interface is used to connect with the power source, and the other end of the electrical interface is connected with the motor driver.

20. A surgical system, comprising:
the robotic arm system according to claim 1;
a navigation system for measuring an orientation of an electric tool; and
a control system, configured to acquire the orientation of the electric tool from the navigation system and drive the robotic arm to move the electric tool to a target position according to a surgical plan.
